(19) United States
(12) Patent Application Publication
(10) Pub. No.: US 2021/0238520 A1
(43) Pub. Date: Aug. 5, 2021

(54) PLANT AND PROCESS FOR THE PRODUCTION OF DESULFURIZED BIOGAS

(71) Applicant: L'Air Liquide, Société Anonyme pour I'Etude et I'Exploitation des Procédés Georges Claude, Paris (FR)

(72) Inventors: Aude BERTRANDIAS, Paris (FR); Solene Valentin, Voreppe (FR)

(21) Appl. No.: 17/157,356

(22) Filed: Jan. 25, 2021

(30) Foreign Application Priority Data
Jan. 31, 2020 (FR) 2000971

(51) Int. Cl. C12M 1/107; C12M 1/06; C12P 5/02; C12M 1/26; B01D 53/22

(52) U.S. Cl. CPC C12M 21/04; C12M 27/02; B01D 53/229; C12M 33/14; C12P 5/023

(57) ABSTRACT

Device for the production of at least partially desulfurized biogas comprising:
a digester,
a means for continuous introduction of biochar into the digester and
a membrane separation unit placed on the stream of biogas exiting from the digester and comprising at least one membrane allowing the sulfur-comprising products to permeate.

### CROSS REFERENCE TO RELATED APPLICATIONS

This application claims the benefit of priority under 35 U.S.C. § 119 (a) and (b) to French Patent Application No. 2000971, filed Jan. 31,2020, the entire contents of which are incorporated herein by reference.

### BACKGROUND

The present invention relates to a plant and to a process for the production of desulfurized biogas or biogas having a reduced content of H₂S.

Biogas is the gas produced during the decomposition of organic matter in the absence of oxygen (anaerobic fermentation), also known as methanization. The decomposition may be natural, as is observed in swamps or in household rubbish dumps; however, the production of biogas can also result from the methanization of waste in a dedicated reactor, the conditions of which are controlled, known as a methanizer or digester, and then in a post-digester, which is similar to the digester and which makes it possible for the methanization reaction to be pushed further.

Biomass will refer to any group of organic matter which can be converted into energy through this methanization process, e.g. treatment plant sludge, manure/liquid manure, agricultural residues, food waste, and the like.

The digester, that is to say the reactor dedicated to the methanization of biomass, is a closed vessel which is or is not heated (operation at a set temperature, between ambient temperature and 55° C.) and the contents of which, constituted by biomass, are stirred, continuously or sequentially. The conditions in the digester are anaerobic and the biogas generated is found in the headspace of the digester (gas headspace), where it is withdrawn. Post-digesters are similar to digesters.

Owing to its main constituents (methane and carbon dioxide), biogas is a powerful greenhouse gas; at the same time, it also constitutes a source of renewable energy, which is appreciable in the context of the increasing scarcity of fossil fuels.

Biogas contains predominantly methane (CH₄) and carbon dioxide (CO₂), in proportions which can vary according to the way in which the biogas is obtained and to the substrate, but can also contain, in smaller proportions, water, nitrogen, hydrogen sulfide (H₂S) or oxygen, and also other organic compounds, in the form of traces, of which H₂S is between 10 and 50 000 ppmv.

Depending on the organic matter which has decomposed and on the techniques used, the proportions of the components differ but, on average, biogas comprises, on a dry gas basis, from 30% to 75% of methane, from 15% to 60% of CO2, from 0% to 15% of nitrogen, from 0% to 5% of oxygen and trace compounds.

Biogas is enhanced in value in various ways. It can, after a gentle treatment, be enhanced in value close to the production site to supply heat, electricity or a mixture of both (cogeneration); the high carbon dioxide content reduces its calorific value, increases the costs of compression and of transportation and limits the economic advantage of enhancing it in value to this nearby use.

More intensive purification of biogas allows it to be more widely used; in particular, intensive purification of biogas makes it possible to obtain a biogas which has been purified to the specifications of natural gas and which can be substituted for the latter; biogas thus purified is known as “biomethane”. Biomethane thus supplements natural gas resources with a renewable part produced within territories; it can be used for exactly the same uses as natural gas of fossil origin. It can feed a natural gas network or a vehicle filling station; it can also be liquefied to be stored in the form of liquefied natural gas (bioLNG), and the like,

Depending on the composition of the biomass, the biogas produced during the digestion contains hydrogen sulfide (H₂S) in contents of between 50 and 50 000 ppm.

Whatever the final destination for enhancement in value of the biogas, it proves to be essential to remove the hydrogen sulfide, which is a toxic and corrosive impurity. Moreover, if the use of the biogas consists in purifying it in order to inject biomethane into the natural gas network, strict specifications limit the amount of H₂S authorized.

Several methods exist for the removal of H₂S and are more or less widespread (beds of activated carbon, addition of iron compounds, physical or chemical absorption, water scrubbing operations, biofilters, and the like), Removal is carried out mainly by adsorption on a bed of activated carbon, outside the digester. In an increasing number of digesters, H₂S reduction is also carried out in part by injection of air/enriched air/O₂ into the gas headspace of the digester, which constitutes an in situ solution. With an injection into the gas headspace at a low dose, solid sulfur is formed from the H₂S and O₂ (eq. (1)), carried out by sulfur-oxidizing bacteria, e.g. Thiobacillus. At a high dose of injected O₂, the mixture is acidified (eq. (2)). Reaction (1) is thus targeted.

H₂S+0.5 O₂→S+H₂O (1)

H₂S+2 O₂→SO₄²⁻+2 H⁺ (2)

The injection amounts of O₂ necessary in practice are different from those expected by the stoichiometry of eq. (1): doses of 0.3%-3% O₂ with respect to the biogas generated are generally recommended, with doses of up to 12% sometimes alluded to.

At present, the in situ injection of air/enriched air/O₂ is not optimized, and the beds of activated carbon thus have to be maintained in order to remove all of the H₂S.

Taking this as starting point, a problem which is posed is that of providing an improved plant promoting the more intensive removal of H₂S.

### SUMMARY

A solution of the present invention is a plant for the production of at least partially desulfurized biogas comprising:
a digester,
a means for continuous introduction of biochar into the digester and
a membrane separation unit placed on the stream of biogas exiting from the digester and comprising at least one membrane allowing the sulfur-comprising products to permeate.

Biochar is activated carbon produced by pyrolysis under limited oxygen conditions of a plant biomass of wood, rice, coconut husk, and the like, type.

Biochar is introduced into the liquid phase of the digester, in order to adsorb the hydrogen sulfide. The adsorption is effective in the presence of oxygen. If the biochar and the oxygen are introduced in satisfactory proportions, all the hydrogen sulfide may be removed.

The biochar which has adsorbed the hydrogen sulfide is discharged at the same time as the digestates and makes it possible to increase the nutritional capacity of the soils of the digestate.

### DESCRIPTION OF PREFERRED EMBODIMENTS

The present invention provides for the combining of the use of biochar inside the digester with a membrane which allows the sulfur-comprising products to permeate, in order to eliminate the use of beds of activated carbons for the purification of the biogas outside the digester. The gaseous permeate will be recycled to the digester, in order to be treated inside the digester by the biochar introduced.

As the case may be, the plant according to the invention can exhibit one or more of the characteristics below:
the digester comprises a mechanical, pneumatic or hydraulic stirring system,
the device comprises a system for extraction and pressing of the digestate produced simultaneously with the biogas,
the device comprises a means for introduction of an oxidation gas making it possible to achieve an oxygen content in the gas headspace of between 0.3% and 3%,
the device comprises, at the outlet of the digester successively in the direction of circulation of the biogas stream, a booster pump, a dryer, a compressor and the membrane separation unit,
the booster pump makes it possible to increase the pressure of the biogas to a pressure of between 1 and 2 bar, preferably between 1 and 1.5 bar, more preferentially still between 1 and 1.3 bar,
the dryer is at a temperature of between 3° C. and 7° C., preferably at 5° C.,
the compressor makes it possible to increase the pressure of the biogas to a pressure of between 5 and 20 bar, preferably between 8 and 15 bar.

Another subject-matter of the present invention is a process for the production of at least partially desulfurized biogas employing a device according to the invention, comprising the following stages:
a) continuous introduction of the biochar into the digester;
b) introduction of the biomass into the digester;
c) anaerobic digestion of the biomass in the digester and production of biogas;
d) separation of the biogas and of the digestate;
e) separation of the sulfur-comprising products contained in the biogas by the membrane route in the membrane separation unit; and
f) recovery of a stream of at least partially desulfurized biogas.

As the case may be, the process according to the invention can exhibit one or more of the characteristics below:
stages a) and b) are carried out simultaneously;
stage a) is carried out before stage b);
the biochar exhibits a particle size of between 1 μm and 6 μm;
the amount of biochar introduced into the digester corresponds at each instant t of the process to a value of between 5% and 15% by weight of the amount of biomass introduced into the digester;
the process comprises, between stages b) and c), a stage of stirring the biochar and the biomass in the digester;
the biomass comprises less than 15% of dry matter;
the biochar has an alkaline pH of greater than 7, preferably of greater than 9, more preferentially still of greater than 11.

The solution according to the invention makes it possible to obtain a biogas stream at the outlet of the membrane separation unit comprising less than 50 ppm of hydrogen sulfide.

The digestate can be used for spreading on the soil.

The invention makes it possible to reduce the cost of purification from hydrogen sulfide of the biogas in an effective way, the reactivity of the oxygen already injected with the sulfur-comprising products being increased by virtue of the introduction of biochar into the liquid part of the digester and the beds of activated carbons treating the gaseous biogas being eliminated by virtue of the permeation of the sulfur-comprising products which remain and of their recycling at the top of the digester.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”), “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

While embodiments of this invention have been shown and described, modifications thereof may be made by one skilled in the art without departing from the spirit or teaching of this invention. The embodiments described herein are exemplary only and not limiting. Many variations and modifications of the composition and method are possible and within the scope of the invention. Accordingly the scope of protection is not limited to the embodiments described herein, but is only limited by the claims which follow, the scope of which shall include all equivalents of the subject matter of the claims.

What is claimed is:

1. A device for the production of at east partially desulfurized biogas comprising:
a digester,
a means for continuous introduction of biochar into the digester and
a membrane separation unit placed on the stream of biogas exiting from the digester and comprising at least one membrane allowing the sulfur-comprising products to permeate.

2. The device according to claim 1, wherein the digester comprises a mechanical, pneumatic or hydraulic stirring system.

3. The device according to claim 1, further comprising a system for extraction and pressing of the digestate produced simultaneously with the biogas.

4. The device according to claim 1, further comprising a means for introduction of an oxidation gas making it possible to achieve an oxygen content in the gas headspace of between 0.3% and 3%.

5. The device according to claim 1, further comprising, at the outlet of the digester successively in the direction of circulation of the biogas stream, a booster pump, a dryer, a compressor and the membrane separation unit.

6. The device according to claim 5, wherein the booster pump makes it possible to increase the pressure of the biogas to a pressure of between 1 and 2 bar, preferably between 1 and 1.5 bar, more preferentially still between 1 and 1.3 bar.

5. The device according to claim 5, wherein the dryer is at a temperature of between 3° C. and 7° C., preferably at 5° C.

5. The device according to claim 5, wherein the compressor makes it possible to increase the pressure of the biogas to a pressure of between 5 and 20 bar, preferably between 8 and 15 bar.

9. A process for production of at least partially desulfurized biogas employing a device according to claim 1, comprising the following stages;
a) continuous introduction of the biochar into the digester;
b) introduction of biomass into the digester;
c) anaerobic digestion of the biomass in the digester and production of biogas;
d) separation of the biogas and of the digestate;
e) separation of sulfur-comprising products contained in the biogas by the membrane route; and
f) recovery of a stream of at least partially desulfurized biogas.

10. The process for the production of biogas according to claim 9, wherein stages a) and b) are carried out simultaneously.

11. The process for the production of biogas according to claim 9, wherein stage a) is carried out before stage b).

12. The process for the production of biogas according to claim 9, wherein the biochar exhibits a particle size of between 1 μm and 6 μm.

13. The process according to claim 9, wherein the amount of biochar introduced into the digester corresponds at each instant t of the process to a value of between 5% and 15% by weight of the amount of biomass introduced into the digester.

14. The process according to claim 9, further comprising, between stages b) and c), a stage of stirring the biochar and the biomass in the digester.

15. The process according to claim 9, wherein the biomass comprises less than 15% of dry matter.

16. The process according to claim 9, wherein the biochar has an alkaline pH of greater than 7, preferably of greater than 9, more preferentially still of greater than 11.
